(19) United States
(12) Patent Application Publication
(10) Pub. No.: US 2024/0245789 A1
(43) Pub. Date: Jul. 25, 2024

(54) POLYMER CONJUGATES OF DRUGS TARGETING 5-HT AND OTHER RECEPTORS IN THE CENTRAL NERVOUS SYSTEM (CNS) THAT ALSO TARGETS RECEPTORS OUTSIDE OF THE CNS

(71) Applicant: Arbormentis LLC, Miami Beach, FL (US)

(72) Inventors: Sara De Martin, Vigonza (IT); Andrea Mattarei, Perarolo di Vigonza (IT); Marco Banzato, Galzignano Terme (IT); Daniela Gabbia, Bassano del Grappa (VI) (IT); Gianfranco Pasut, Padova (IT); Paolo L. Manfredi, Miami Beach, FL (US)

(21) Appl. No.: 18/559,579

(22) PCT Filed: May 10, 2022
(86) PCT No.: PCT/US2022/028559

§ 371 Date: Nov. 8, 2023

Related U.S. Application Data
(60) Provisional application No. 63/364,364, filed on May 9, 2022.
(60) Provisional application No. 63/186,298, filed on May 10, 2021.

(51) Int. Cl. A61K 47/60; A61P 1/16

(52) U.S. Cl. CPC A61K 47/60; A61P 1/16

(57) ABSTRACT

Psychedelic drug (PD) polymer conjugates having a general structure PD-(X-Poly-T)n, wherein PD is a CNS active psychedelic drug targeting serotonergic receptors, with possible affinity also for other receptors. (X-Poly-T) is, independently for each occurrence, hydrogen or the moieties are, independently: X is a stable (enzymatically and/or hydrolytically under physiological conditions) linker comprising a covalent bond or a chain of atoms that covalently attaches a small molecule 5-HT receptors agonist drug moiety to the Poly derivative. Poly is a covalently bonded chain of repeating monomer units that form a polymer or an oligomer backbone of synthetic or natural origin. T is terminal group of Poly and is represented by any suitable chemical group which, depending upon preference, is unreactive or reactive with other chemical moieties, or has a targeting property. N is an integer comprised between 1 and 6.

### CROSS-REFERENCE TO RELATED APPLICATION

This application claims priority to, and the benefit of the filing date of, U.S. Patent Application Ser. No. 63/186,298, filed on May 10, 2021, and also claims priority to, and the benefit of the filing date of, U.S. Patent Application Ser. No. 63/364,364, filed on May 9, 2022 the disclosures of which are incorporated by reference herein in their entireties.

### FIELD OF THE INVENTION

Aspects of the present invention generally relate to polymer conjugates of psychedelic drugs, and the use of the therapeutic and preventative aspects of same on the immune system, the metabolic system, and the visual system.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Serotonin (5-HT) is a neurotransmitter with known actions in the central nervous system (CNS). 5-HT receptor modulators are also known as psychedelic compounds and some of these compounds are under development as candidate drugs for depression or other CNS diseases. However, serotonin receptor modulating drugs also target extra CNS receptors, with downstream effects that have potential therapeutic value against inflammation and lipid dysregulation. The inventors previously disclosed potential peripheral (out of the CNS) therapeutic effects of 5-HT agonists in International Patent Application No. PCT/US2020/021400. However, these drugs also have central effects. Therefore, preferential targeting of these peripheral 5-HT receptors by serotonergic drugs with restricted access to the CNS is a potential novel strategy to exert peripheral serotonin receptor modulating therapeutic effects while avoiding CNS effects, including psychedelic effects. This aim is here pursued by acting on the capability of these drugs to cross the blood-brain barrier (BBB), an anatomic-functional barrier effective at eliminating or reducing the passage of select xenobiotics into the brain. The present inventors designed polymer-drug conjugates (PDCs) of CNS acting psychedelic drugs (PDs) to impede, decrease, or modulate their crossing of the BBB. For certain inflammatory diseases of the gastrointestinal system there may also be an advantage in modulating access across the intestinal barrier (IB). Potentially therapeutic peripheric effects of PDCs of molecules known as PDs (PDCs of PDs) could become more advantageous in the absence or with reduced BBB and IB crossing, leading to concomitant down-modulation of CNS effects. In fact, these PDCs of PDs cannot cross the BBB or have modulated or limited BBB crossing capabilities, because of specific features of the polymer structure, their molecular weight and or their hydrodynamic volume and the chemical-physical properties. The coupling of a PD with a specific tailored polymer results in novel molecules with a favorable risk-benefit ratio and improved pharmacokinetic and pharmacodynamic profile for select diseases and disorders. In summary, this invention obtains PDCs of PDs with the intent to preferentially target 5-HT receptors located outside the CNS for the treatment of diseases, disorders and conditions linked to unbalanced activity of 5-HT peripheral receptors.

CNS psychoactive drugs, including PDs, cross the BBB and reach receptors in the brain, including 5-HT2A receptors, and exert certain central effects, including psychoactive side effects and potentially therapeutic psychoactive effects. The central effects of PDs are primarily caused by their binding to serotoninergic receptors, including the 5-HT2A isoform and the 5-HT2C isoform located on the membrane of neurons in the brain. These drugs may have prominent psychoactive effects, including psychedelic and dissociative effects. Small molecules such as for example phenylalkylamines, have some degree of selectivity for 5-HT receptors and cause psychedelic effects when given at appropriate doses. Phenethylamines molecules, including psilocybin, administered in single or multiple sessions, are under clinical investigation for a multiplicity of psychiatric diseases and symptoms. Depression, anxiety, end of life angst, and addiction are some of the psychiatric diseases and symptoms that may be improved by psychedelics [Kvam T M, Stewart L H, Andreassen O A. Psychedelic drugs in the treatment of anxiety, depression and addiction. Tidsskr Nor Laegeforen. 2018 Nov. 12; 138(18)]. The subacute impact of psilocybin on brain function has recently been assessed in two clinical trials of depression (MR/J00460X/1, NCT03429075), demonstrating efficacy-related brain changes, which correlate with robust antidepressant effects across the two studies, suggesting that the global increases in brain network integration is an antidepressant mechanism for psilocybin therapy (Daws at al., Increased global integration in the brain after psilocybin therapy for depression. Nature Medicine, 2022).

Despite this renovated interest of the scientific psychiatric community in 5-HT2A agonists for the treatment of psychiatric indications, the recreational abuse of these substances and drugs poses a significant barrier to their development as pharmaceuticals. Strong public safety and regulatory concerns remain about the use of substances with the potential for inducing psychedelic effects as treatment of diseases. Therefore, these psychoactive substances are presently illegal in most countries, including the United States of America, and their development into potentially therapeutic agents has been hampered. In the USA and in many other countries, natural and synthetic psychedelic substances are classified as schedule I substances characterized by high abuse potential and no regulatory approved clinical uses, despite their relative safety and low addiction potential, underscored in recent scientific publications (Brown R T, Nicholas C R, Cozzi N V, Gassman M C, Cooper K M, Muller D, Thomas C D, Hetzel S J, Henriquez K M, Ribaudo A S, Hutson P R. Pharmacokinetics of Escalating Doses of Oral Psilocybin in Healthy Adults. Clin Pharmacokinet. 2017 December; 56 (12):1543-1554; Studerus E, Kometer M, Hasler F, Vollenweider F X. Acute, subacute and long-term subjective effects of psilocybin in healthy humans: a pooled analysis of experimental studies. J Psychopharmacol. 2011 November; 25(11):1434-52; Johnson M W, Griffiths R R, Hendricks P S, Henningfield J E. The abuse potential of medical psilocybin according to the 8 factors of the Controlled Substances Act. Neuropharmacology. 2018 November; 142:143-166). In summary, the development of psychedelic substances for the treatment of diseases remains problematic due to the potent central effects of these drugs, which can be modulated only by dose reduction, with potential loss of efficacy for neuropsychiatric disorders and other disorders.

The current consensus is that the psychoactive effects of psychedelic drugs are necessary for their therapeutic effects against neuropsychiatric disorders.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

The present invention discloses psychedelic drugs polymer conjugates leading to therapeutic advantages over psychedelic drugs. The chemically modified psychedelic drugs described herein have applications in the fields of drug discovery and pharmacotherapy, polymer chemistry, and others. Of particular interest are therapeutic and preventive effects of these new molecular entities on the immune system, the metabolic system, and the visual system.

Aspects of the present invention are directed to psychedelic drug (PD) polymer conjugates having a general structure PD-(X-Poly-T)n, wherein PD is a CNS active psychedelic drug targeting serotonergic receptors, with possible affinity also for other receptors. PD has at least one chemically reactive functional group (e.g., a primary amine or secondary amine, hydroxyl, sulfhydryl, carboxyl, aldehyde or ketone), or (if absent) this group can be chemically introduced, pendant thereto chemically reacted to the linker to form a covalent bond. N is an integer comprised between 1 and 6.

(X-Poly-T) is, independently for each occurrence, hydrogen or the moieties are, independently for each occurrence, as follows:

X is a stable (enzymatically and/or hydrolytically under physiological conditions) linker comprising a covalent bond or a chain of atoms that covalently attaches a small molecule 5-HT receptors agonist drug moiety to the Poly derivative. Examples of linkers disclosed include but are not limited to the following: carboxylate ester, phosphate ester, anhydride, acetal, ketal, acyloxyalkyl ether, imine, hydrazone, carbohydrazone, carbamate, peptides, nucleotides, C—C bond (e.g., in aliphatic chain), ether, amide, oxime, enamine, semicarbazone, semicarbazide, thioether.

Poly is a covalently bonded chain of repeating monomer units that form a polymer or an oligomer backbone of synthetic or natural origin. Examples of Poly backbones disclosed include but are not limited to the following: poly(ethylene glycol) (PEG), poly(N-vinylpyrrolidone), N-hydroxy-ethyl methacrylamide copolymer, poly(2-ethyl-2-oxazoline), poly(N-acryloylmorpholine), poly(propylene glycol), poly(vinyl alcohol), polyglutamic acid, hyaluronic acid, or polysialic acid or other polysaccharides. Poly has a preferred average molecular weight between 80 and 40000 Da, preferably at least 100 Da, more preferably at least 200 Da. In some preferred embodiments of the invention, Poly is a derivative of poly(ethylene glycol) (PEG), of linear or branched structure, mono-, bi-functional or heterobifunctional, with an average molecular weight between 120 and 40000 Da. Some preferred Poly are PEG-O-163 Da, PEG-COO-207 Da, PEG-O-251 Da, PEG-O-295 Da, PEG-O-339 Da, PEG-O-383 Da, PEG-O-427 Da, PEG-O-471 Da, PEG-O-515 Da, PEG-O-559 Da.

T is terminal group of Poly and is represented by any suitable chemical group which, depending upon preference, is unreactive or reactive with other chemical moieties, or has a targeting property. Examples of terminal groups disclosed include but are not limited to the following: hydroxyl, amino, sulfide, carboxy, cyano, optionally substituted aryloxy, lower alkoxy (e.g., methoxy, ethoxy, propoxy, or butoxy), aryl, lower alkyl, lower alkenyl, lower alkynyl, cycloalkyl, halogen atom (e.g., fluorine, chlorine, bromine, iodine), tosylate, mesylate, isocyanate, hydrazine, azide, maleimide, orthopyridyl disulfide, N-succinimidyloxy, sulfo-N-succinimidyloxy, 1-benzotriazol, 1-imidazolyloxy, p-nitrophenyloxy, formyl.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, explain the principles of the present invention.

FIGS. 1A-1D are graphs showing the effect of psilocin on lipid accumulation in HepG2 cells. *p<0.05, **p<0.01, ***p<0.001, ****p<0.0001 vs control cells; #p<0.05, ##p<0.01 vs PA:OA mix.

FIG. 2 are microphotographs showing staining of triglycerides (in green) in HepG2 cells in the experimental conditions described above. Cell nuclei are stained blue with DAPI.

FIGS. 3A and 3B are graphs showing mRNA expression of CCL2 and TNF-α in HepG2 cells.

FIGS. 4A-4D are graphs showing body weight and body weight increase, and daily and water food intake in two study groups.

FIG. 5 includes microphotographs showing normal hepatic parenchyma in mice fed with normal diet with or without psilocyn daily treatment (left), and reduction of lipid accumulation (in red) in mice with NAFLD treated daily with psilocybin (right).

FIGS. 6A and 6B are graphs showing protein expression of PLIN2 and NMDAR1 in the liver. *p<0.05, ***p<0.001 vs controls, #p<0.05 vs mice with NAFLD.

FIG. 7 is a schematic showing the synthesis of PSI-TetraEGME and PSI-HexaEGME using the strategy proposed in this specification.

FIG. 8 is a graph showing the 1H NMR spectrum of 2-(4-((2,5,8,11-tetraoxatridecan-13-yl)oxy)-1H-indol-3-yl)-N,N-dimethylethan-1-aminium trifluoroacetate (PSI-TetraEGME trifluoroacetate).

FIG. 9 is a graph showing the 13C NMR spectrum of 2-(4-((2,5,8,11-tetraoxatridecan-13-yl)oxy)-1H-indol-3-yl)-N,N-dimethylethan-1-aminium trifluoroacetate (PSI-TetraEGME trifluoroacetate).

FIG. 10 is a graph showing the 1H NMR spectrum of 2-(4-((2,5,8,11,14,17-hexaoxanonadecan-19-yl)oxy)-1H-indol-3-yl)-N,N-dimethylethan-1-amine (PSI-HexaEGME).

FIG. 11 is a graph showing the 13C NMR spectrum of 2-(4-((2,5,8,11,14,17-hexaoxanonadecan-19-yl)oxy)-1H-indol-3-yl)-N,N-dimethylethan-1-amine (PSI-HexaEGME).

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Aside from CNS receptors, psychedelic drugs also target serotoninergic receptors located outside the CNS, with pharmacodynamic effects that may have potential therapeutic value. These peripheral effects are generally offset by the CNS effects of these drugs. Therefore, targeting these peripheral 5-HT receptors by restricting their access to the CNS is a potential novel therapeutic options that prevents psychedelic side effects while maintaining potentially therapeutic peripheral serotonin-system modulating effects. This objective of targeting preferentially peripheral serotonin receptors can be pursued by modulating the crossing of the BBB. Physiologically, the BBB protects the brain from toxic molecules. The BBB allows and even modulates the passage of essential nutrients and selected substances and is effective at eliminating or decreasing the passage of xenobiotics, regulating the rate at which some substances reach brain tissue. PDCs of CNS acting PDs may impede, decrease, or modulate the crossing by PDs of the BBB. Potentially therapeutic effects of PDCs of molecules know as psychedelics could become advantageous in the absence or with reduced CNS effects, by improving the safety window of these drugs while maintaining or even improving their therapeutic effects. PDCs that cannot cross the BBB or have modulated or limited BBB or IB crossing capabilities can preferentially exert peripheral actions that are potentially therapeutic without central side effects.

Although the current studies of PDs, including 5-HT2A agonists such as psilocybin, are mainly centered at the resolution of CNS pathologies, such as depression and other psychiatric disorders resistant to other drug treatments, 5-HT receptors may have a role in the pathogenesis and potential treatment of other diseases, including peripheral diseases, due to the presence of these receptors on peripheral organs.

In summary, 5-HT modulators also target extra-CNS receptors, thereby exerting potential therapeutic peripheral effects. For example, achieving peripheral anti-inflammatory effects (see Example 1), metabolic effects (e.g., hepatic effects as described in Example 1) and other therapeutic effects, including effects on select cells of the retina (Steuer H, Jaworski A, Elger B, et al. Functional characterization and comparison of the outer blood-retina barrier and the blood-brain barrier. Invest Ophthalmol Vis Sci. 2005; 46(3):1047-1053. doi:10.1167/iovs.04-0925), could be advantageous with these novel PDCs of PDs. In addition, by avoiding or limiting access to CNS, the dose of these novel molecules can be augmented to increase peripheral efficacy without causing psychedelic effects mediated by binding to CNS receptors.

In order to best benefit from extra-CNS effect and/or avoid CNS effects, serotonin modulating drugs, including psilocybin, can be modified by the covalent conjugation of polymers, such as for example polyethylene glycol (PEG, PEGylation) and other polymers so to modulate or impede BBB crossing, according to the size and the characteristics of the polymer chain.

With this invention the present inventors aim at obtaining PDCs of PDs binding to 5-HT receptors and possibly other receptors, with the intent to preferentially target peripheral (i.e., extra-CNS) receptors targeted by these drugs for the treatment of diseases, disorders and conditions linked to unbalanced activity of peripheral receptors, preferentially. Additionally, these novel PDCs of PDs may bind to and modulate the activity of other receptors, in addition to 5-HT2A receptors, preferentially outside the CNS, with therapeutic potential.

Furthermore, the activity of these novel drugs at 5-HT or other receptors in the CNS may not be completely abolished by polymer conjugation of the drug, but may simply be modulated, and polymer conjugation of these drugs may thus result in a more favorable pharmacodynamic or pharmacokinetic profile at both central and peripheral receptors. For example, PEGylated-based platforms can also be exploited to optimize and enhance the brain delivery of molecules characterized by a poor BBB penetration (Fernandes et al., Bioconjugate Chem, 2018, 29, 1677-1689). Thus, these PDCs drugs may result in therapeutic effects for both CNS and extra-CNS conditions that represent an improvement in the efficacy/safety ratio of the parent molecule.

In some cases, these molecules may have reduced/abolished intestinal absorption according to the size/feature of the coupled polymeric chain, and these molecules will preferentially target intestinal receptors, being useful for the treatment of inflammatory diseases of the gastrointestinal tract, such as inflammatory bowel diseases, including ulcerative colitis and Chron's disease and irritable bowel syndrome.

On the basis of desired therapeutic effects, it could be advantageous to maintain some degree of central 5-HT receptor activity, or it can be preferable to generate increasingly selective peripheral modulators of 5-HT receptors and other receptors. The BBB penetration will be modified, and the intestinal penetration may be modified according to the desired therapeutic use of these novel molecules (PDCs of PDs).

Diseases and disorders where inflammation concurs in triggering or maintaining the pathological process are of relevance (COVID-19 and other infections, ARDS, DIC, inflammatory bowel disease, NAFLD, NASH, diabetes, atopic dermatitis, and asthma are examples of such diseases). As described above, alterations in 5-HT signaling have been described in inflammatory conditions of the gut, such as inflammatory bowel disease, and the liver, in metabolic disorders such as non-alcoholic fatty liver disease (NAFLD) and steatohepatitis (NASH) and diseases of the eye (see International Patent Application No. PCT/US2020/021400). NAFLD is a condition that affects more than 200 million people worldwide and is considered the hepatic manifestation of metabolic syndrome. Approximately 10-20% of individuals affected by NAFLD progress over time from uncomplicated steatosis to NASH. NASH is a chronic liver disease which, besides the presence of hepatocellular lipids, presents inflammation and injury of the hepatic parenchyma. In this peculiar microenvironment, the activation of innate and adaptive immune cells, in combination with increased metabolites and endoplasmic reticulum stress, could lead to a cycle of hepatic necro-inflammation and regeneration potentially leading to the development of hepatocellular carcinoma (HCC), a primary liver tumor with extremely limited therapeutic options. NASH has become an emerging risk factor for HCC, and the prevalence of this etiology is predicted to increase in the next years. Furthermore, it has recently been demonstrated that NASH-HCC patients might be less responsive to immunotherapy, probably owing to NASH-related aberrant T cell activation that leads to impaired immune surveillance (Pfister et al. NASH limits anti-tumor surveillance in immunotherapy-treated HCC. Nature 2021; 592: 450-456). Of note, although the intensive research in this field, no drug had gained approval for NAFLD or NASH. Therefore, the finding of cure for NASH is an urgent medical need (Karlsen et al., The EASL-Lancet Liver Commission: protecting the next generation of Europeans against liver disease complications and premature mortality. Lancet 2022; 399: 61-116). An early pharmacological intervention in NAFLD/NASH patients could be a strategy to prevent disease progression or cure the disease, restore liver function, and finally prevent HCC development. NAFLD and NASH are the hepatic manifestation of the metabolic syndrome. In the USA, the metabolic syndrome prevalence increased from 1988 to 2012 for every sociodemographic group; by 2012, more than a third of all US adults met the definition and criteria for metabolic syndrome agreed to jointly by several international organizations. (Moore J X, Chaudhary N, Akinyemiju T. Metabolic Syndrome Prevalence by Race/Ethnicity and Sex in the United States, National Health and Nutrition Examination Survey, 1988-2012. Prev Chronic Dis 2017; 14:160287. DOI: http://dx.doi.org/10.5888/pcd14.160287) The metabolic syndrome is associated with cardiovascular disease, obesity, arthritis, NAFLD, NASH, MDD, schizophrenia, dementia, and cancer (Colognesi et al., Biomedicines, 2020). Metabolic disorders that may be treated or prevented by 5HT-2A modulating drugs substances and drugs include: the metabolic syndrome, obesity, hyperglycemia, type 2 diabetes mellitus, high blood pressure, coronary artery disease including myocardial infarction and unstable angina, NAFLD and NASH, hypogonadism, testosterone insufficiency, hypothalamic-pituitary axis disorders, and BDNF insufficiency, including WAGR syndrome, 11p deletion, and 11p inversion, and Prader-Willi, Smith-Magenis, and ROHHAD syndromes.

Our results (as shown in Example 1) suggest that 5-HT2A agonists, e.g., low chronic doses of psilocybin may improve liver steatosis and reduce inflammation. Based on our experimental findings, 5-HT2A drugs may have strong therapeutic potential for the treatment of the metabolic syndrome, not only as an appetite suppressant and anti-obesity drug, but as a potentially disease modifying treatment with actions and influences at the molecular level on hepatocytes and Langerhans cells also by modulating the expression of other receptors, such as for example NMDAR in the liver. The present inventors demonstrated that in vitro psilocin could reduce the uptake of lipids by hepatocyte-like cells, and has a modulating effect on lipid transporters, such as adipose differentiation-related protein (PLIN-2). This effect was accompanied by a reduction of inflammation. The same effect could be observed in vivo in an experimental model of NAFLD obtained by the administration of a diet rich in fat to c57BL6 mice, treated with low doses of oral psilocybin by oral gave once a day. These mice had a reduction in body weight gain, but not of their food intake when compared to controls, in the absence of evident psychedelic effects. Based on these studies, the present inventors concluded that targeting NAFLD/NASH and the metabolic syndrome and related pathologies with serotoninergic agonist molecules with preferential peripheral actions may be a promising strategy.

### EXAMPLES

### Example 1: 5-HT Agonists Reduce Liver Steatosis and Inflammation In Vitro and In Vivo

### In Vitro Study

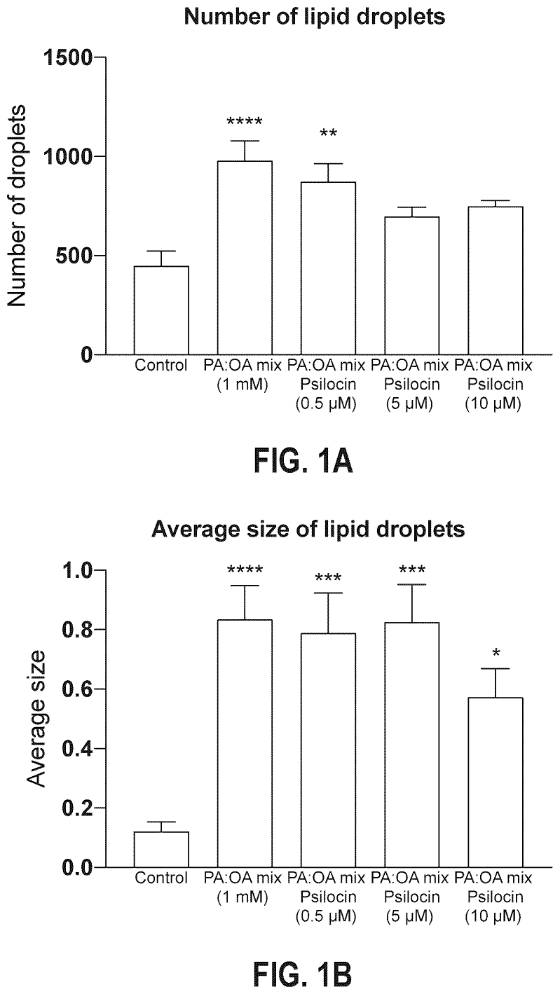

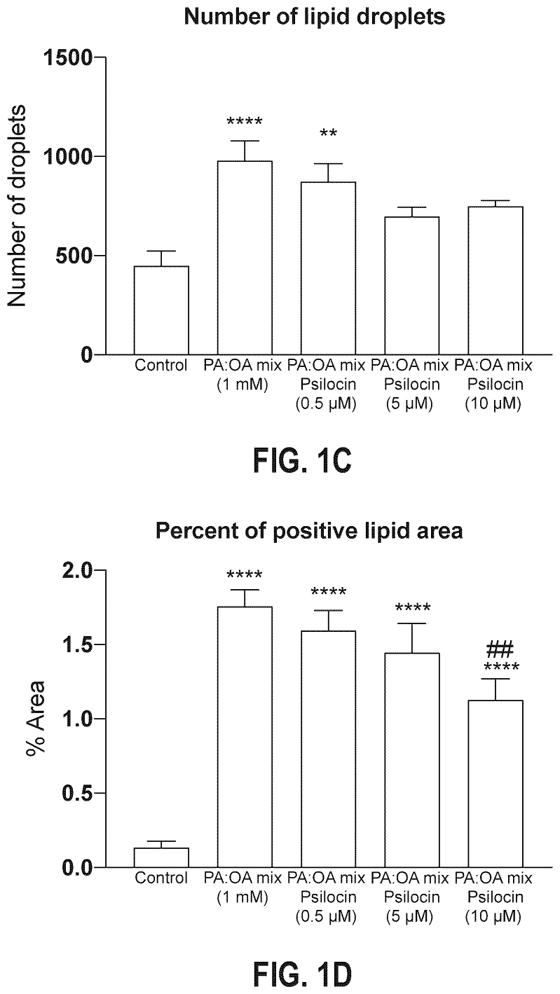

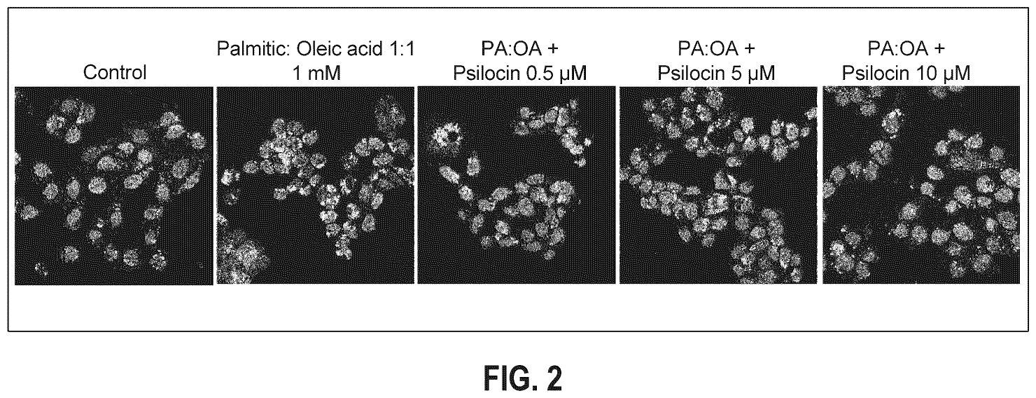

The present inventors used the hepatocyte-like hepatoma cell line HepG2 to set up an experimental in vitro model of NAFLD, obtained by incubating the cells for 24 hours with a 1 mM mixture of 1:1 paltmitic acid (PA):oleic acid (OA) (Movavcova et al., The effect of oleic and palmitic acid on induction of steatosis and cytotoxicity on rat hepatocytes in primary culture. Physiol Res 2015; 64(Suppl 5):S627-36). FIGS. 1A-1D and 2 show that psilocin reduces the accumulation of triglycerides inside the cells in a dose-dependent manner, causing a reduction of the number and the area of lipid droplets.

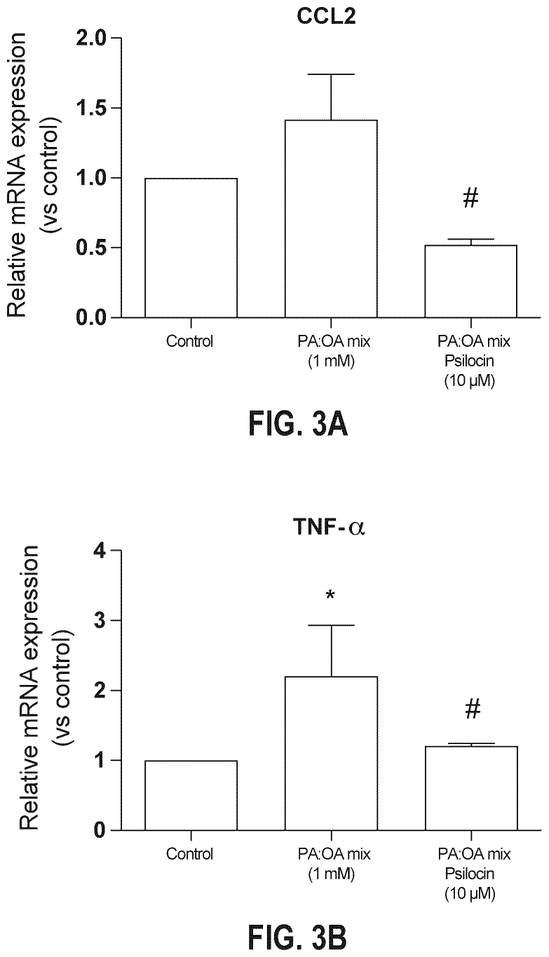

Interestingly, this effect on lipid accumulation is accompanied by a reduction in the mRNA expression of the 2 inflammatory cytokines CCL-2 and TNF-α, which play pivotal roles in liver inflammation as shown in FIGS. 3A and 3B.

### In Vivo Study

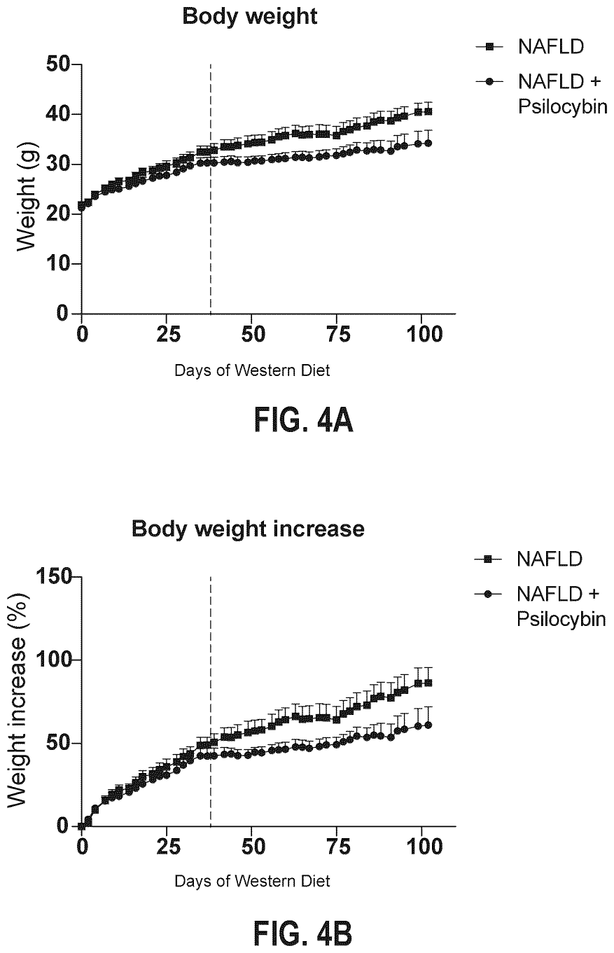

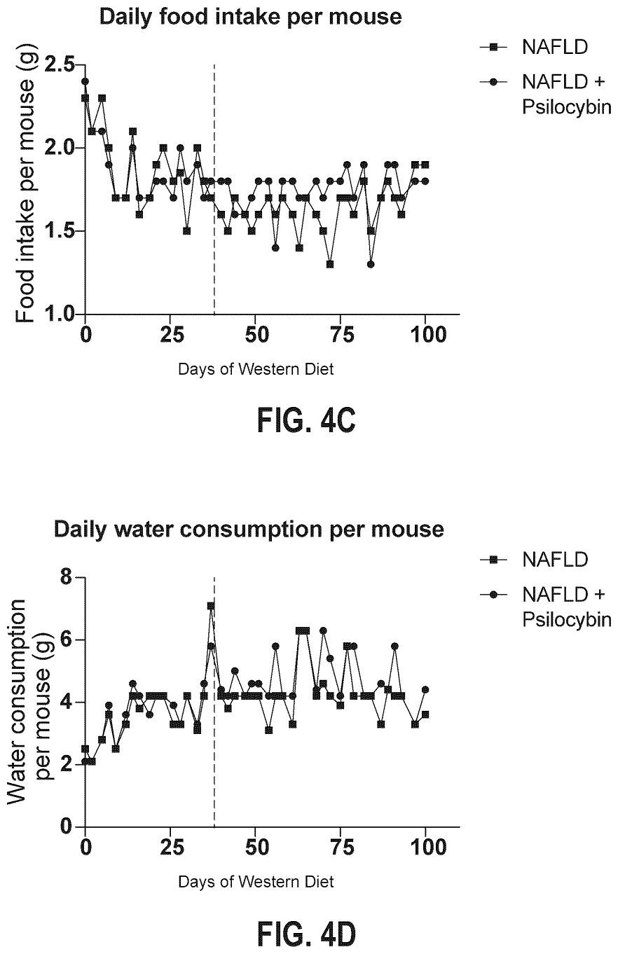

The present inventors tested the effect of psilocybin in mice with non alcoholic fatty liver disease (NAFLD) obtained by the administration of a diet rich in fat (60% kcal from fat) boosted with 30% fructose in drinking water (n=6 animals per group). Psilocybin was administered daily by oral gavage (0.05 mg/kg). Interestingly, mice treated with psilocybin showed a significant reduction of body weight gain with respect to untreated mice with NAFLD, but this was not due to a reduction of the appetite, since the food and water intake was very similar in the two study groups (FIGS. 4A-4D).

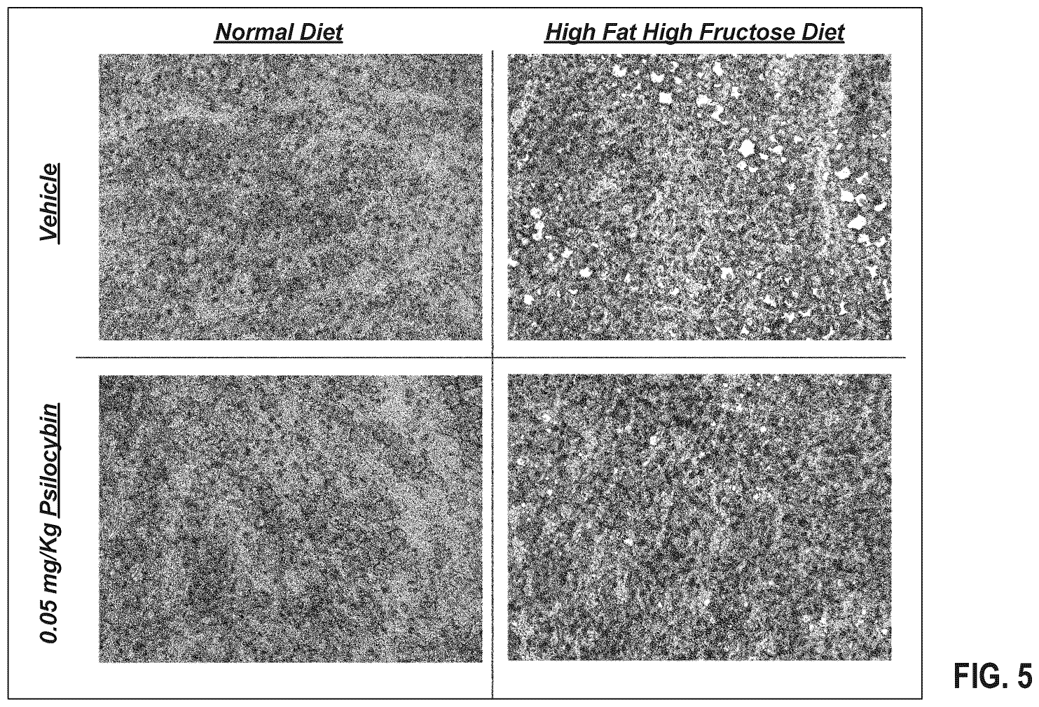

The histological analysis performed with the Sirius O red staining at sacrifice demonstrated that mice with NAFLD treated with psilocybin had a reduction of the presence of lipids in the liver. The lipid droplets were less and smaller that those present in their untreated counterparts. Interestingly, no signs of hepatic damage were observed in a small (n=4) mice fed with standard diet and treated daily with 0.05 mg/kg of psilocybin, indicating that the serotoninergic alcaloid had displayed no hepatotoxicity in our experimental conditions. No signs of psychoactive effects have been observed during the weeks of treatment (FIG. 5).

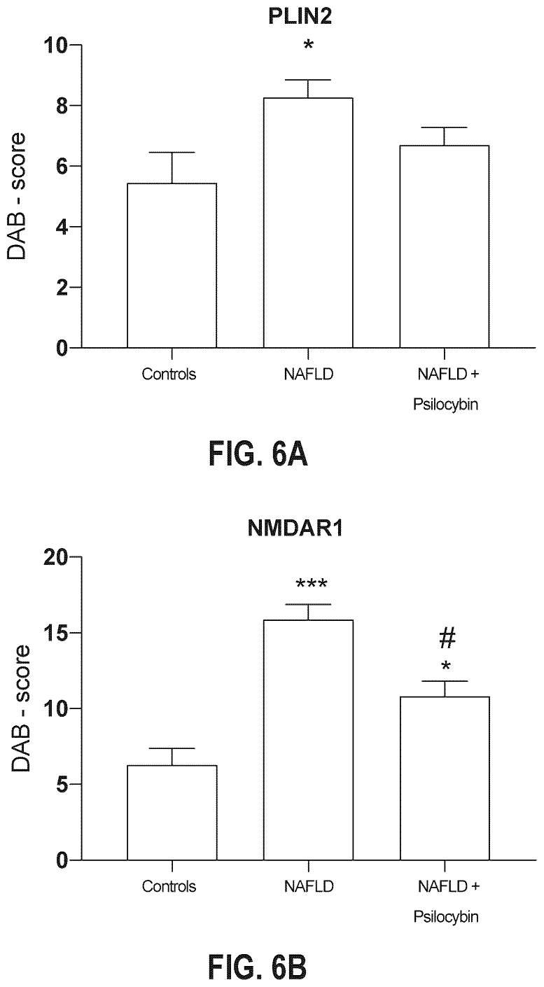

As shown in FIGS. 6A and 6B, the present inventors also demonstrated in the same in vivo model that psilocybin treatment can modulate the protein expression of the lipid transporter called adipose differentiation-related protein 2 (PLIN-2), which is involved in the storage of fatty acids inside the lipid droplets, evaluated by immunohistochemistry. In particular, psilocybin treatment decreased the expression of this transporter, suggesting that this modulating effect can at least in part be pivotal for the reduction of the accumulation of triglycerides. Furthermore, psilocyn was also able to modulate the expression of the NMDAR1 receptor, whose role in the liver is still far to be completely understood, counteracting its overexpression induced by steatosis. These data suggest that complex signaling pathways are involved in the mechanism of action by which serotoninergic compounds such as psilocybin display their peripheral effects.

The present inventors are here disclosing PDCs of psychedelic drugs with 5-HT receptors modulating actions and/or other actions preferentially at extra CNS receptors. The present inventors are also disclosing the PDCs adopting polymers which can modulate/reduce/eliminate the ability of the parent drug to cross the BBB and or the intestinal barrier. The present inventors are developing molecules able to cross the intestinal cells and reach the portal circulation and the liver because novel PDCs with potential therapeutic activity for liver conditions are of particular interest. The present inventors are developing PDCs that can be administered, preferably but not exclusively, via the oral pathway. Oral administration is one of the preferred routes of administration and is the most common route of administration for small molecule drugs. The polymer chain length and features can be modulated to maintain the desired intestinal absorption for the targeted disease, disorder or condition. The desired therapeutic activity may be restricted to the gastrointestinal tract and therefore gastrointestinal (GI) crossing is restricted. If the desired effects are not restricted to the GI tract and the novel molecule is intended for other peripheral organs, the PDCs of PDs are designed to cross the GI barrier while the BBB is downmodulated. Finally, if the target is the brain, PDCs may also be designed to modulate BB crossing in the desired way.

Examples of 5-HT receptors agonists and partial agonists of interest are: psilocin, norpsilocin, 4-hydroxytryptamine, N,N-dimethyltryptamine, N-methyltryptamine, tryptamine, psilocybin, baeocystin, norbaeocystin, (R)- and (S)-2,5-Dimethoxy-4-iodoamphetamine (DOI), lysergic acid diethylamide (LSD), lysergic acid ethylamide (Deethyl-LSD), ibogaine, noribogaine (see Master file below).

Examples of disclosed PDCs of PDs (also shown in Table 1, below) polymer conjugates have the general structure:

PD-(X-Poly-T)ₙ

wherein PD is a CNS active psychedelic drug targeting serotonergic receptors, with possible affinity also for other receptors. PD has at least one chemically reactive functional group (e.g., a primary amine or secondary amine, hydroxyl, sulfhydryl, carboxyl, aldehyde or ketone), or if absent this group can be chemically introduced, pendant thereto chemically reacted to the linker to form a covalent bond.

n is an integer comprised between 1 and 6 and -(X-Poly-T) is, independently for each occurrence, hydrogen or the moieties are, independently for each occurrence, as follows:

X is a stable (enzymatically and/or hydrolytically under physiological conditions) linker comprising a covalent bond or a chain of atoms that covalently attaches a small molecule 5-HT receptors agonist drug moiety to the Poly derivative. Examples of linkers disclosed include but are not limited to the following: carboxylate ester, phosphate ester, anhydride, acetal, ketal, acyloxyalkyl ether, imine, hydrazone, carbohydrazone, carbamate, peptides, nucleotides, C—C bond (e.g., in aliphatic chain), ether, amide, oxime, enamine, semicarbazone, semicarbazide, thioether.

Poly is a covalently bonded chain of repeating monomer units that form a polymer or an oligomer backbone of synthetic or natural origin. Examples of Poly backbones disclosed include but are not limited to the following: poly(ethylene glycol) (PEG), poly(N-vinylpyrrolidone), N-hydroxy-ethyl methacrylamide copolymer, poly(2-ethyl-2-oxazoline), poly(N-acryloylmorpholine), poly(propylene glycol), poly(vinyl alcohol), polyglutamic acid, hyaluronic acid, or polysialic acid or other polysaccharides. Poly has a preferred average molecular weight between 80 and 40000 Da, preferably at least 100 Da, more preferably at least 200 Da. In some preferred embodiments of the invention, Poly is a derivative of poly(ethylene glycol) (PEG), of linear or branched structure, mono-, bi-functional or heterobifunctional, with an average molecular weight between 120 and 40000 Da. Some preferred Poly are PEG-O-163 Da, PEG-COO-207 Da, PEG-O-251 Da, PEG-O-295 Da, PEG-O-339 Da, PEG-O-383 Da, PEG-O-427 Da, PEG-O-471 Da, PEG-O-515 Da, PEG-O-559 Da.

T is terminal group of Poly and is represented by any suitable chemical group which, depending upon preference, is unreactive or reactive with other chemical moieties, or has a targeting property. Examples of terminal groups disclosed include but are not limited to the following: hydroxyl, amino, sulfide, carboxy, cyano, optionally substituted aryloxy, lower alkoxy (e.g., methoxy, ethoxy, propoxy, or butoxy), aryl, lower alkyl, lower alkenyl, lower alkynyl, cycloalkyl, halogen atom (e.g., fluorine, chlorine, bromine, iodine), tosylate, mesylate, isocyanate, hydrazine, azide, maleimide, orthopyridyl disulfide, N-succinimidyloxy, sulfo-N-succinimidyloxy, 1-benzotriazol, 1-imidazolyloxy, p-nitrophenyloxy, formyl.

In the following descriptions, the synthesis of PEG derivatives of psilocin by connecting a short PEG chain (oligo(ethylene glycol) n=4, 6) to the 4-hydroxyl group of psilocin is disclosed.

PDCs of psilocin and other 5-HT receptors active drugs, prevent the drugs from crossing the BBB, allowing activity on 5-HT receptors and potentially other receptors at a peripheral level without CNS effects. This may be advantageous when the intended use is treatment of diseases that may benefit from the engagement of peripheral (extra-CNS) rather than central (CNS) receptors.

Different linkages between PD and Poly and different T moieties of Poly are disclosed to modulate the pharmacokinetic/pharmacodynamic parameters of the resulting PD-(X-Poly-T)ₙ.

TABLE 1

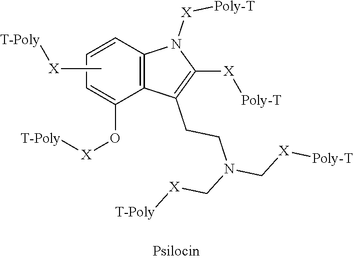

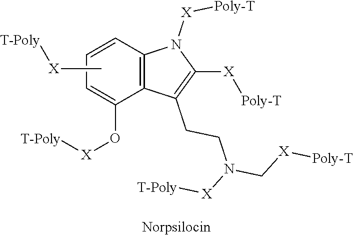

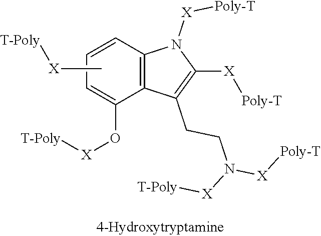

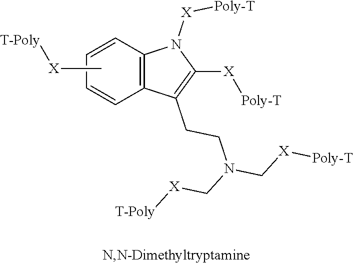

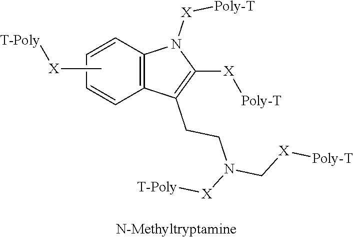

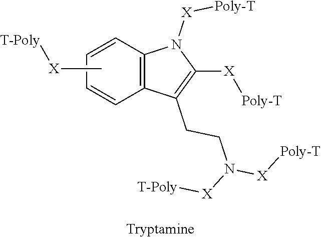

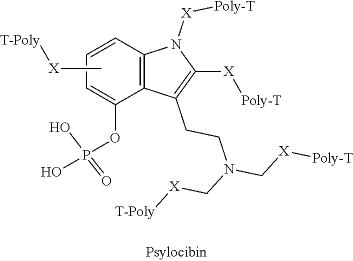

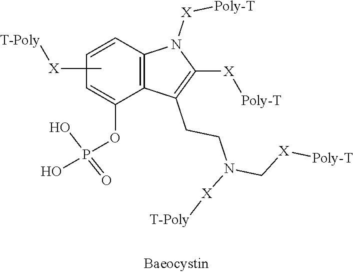

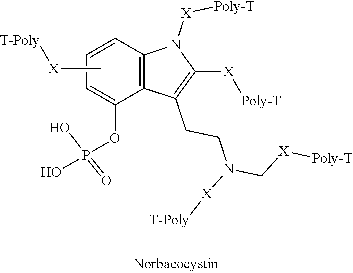

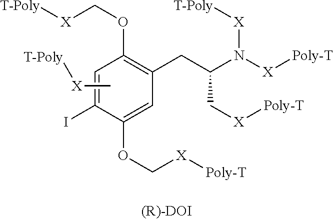

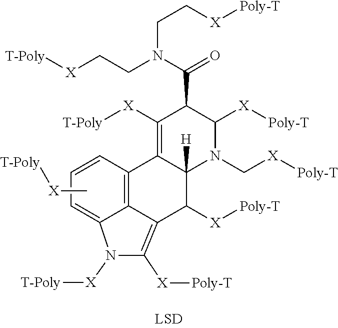

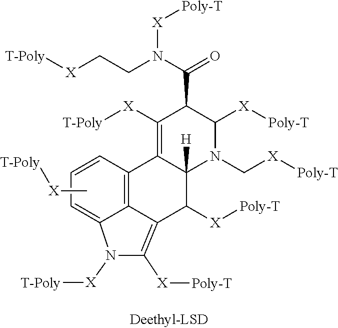

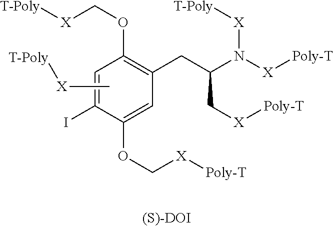

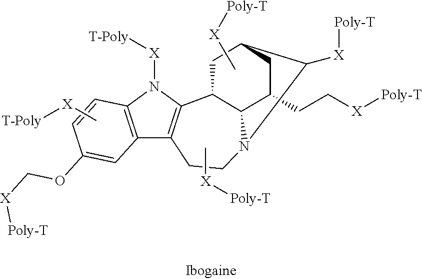

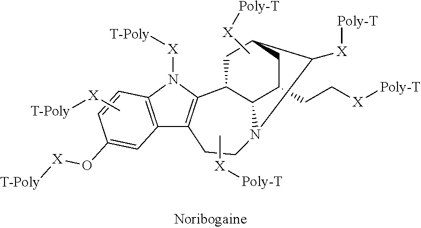

### Example 2: 2-(4-((2,5,8,11-tetraoxatridecan-13-yl)oxy)-1H-indol-3-yl)-N,N-dimethylethan-1-amine (PSI-TetraEGME)

The compound structures in the examples below were confirmed by one or more of the following methods: ¹H NMR, ¹³C NMR, mass spectrometry, HPLC-UV. ¹H NMR spectra were determined using an NMR spectrometer operating at 300 MHz or 400 MHz field. Chemical shifts were referenced to signals from residual protons in deuterated chloroform as follows: CDCl₃=7.25 ppm. Peak multiplicities are designated as follows: s, singlet; d, doublet; t, triplet; and m, multiplet. Coupling constants are given in Hertz (Hz).

Mass spectra (MS) data are obtained using a mass spectrometer with ESI ionization and Ion trap or TOF mass analyzer.

In this and other examples herein, reagents and solvents may be purchased from commercial suppliers, and may be used without further purification unless otherwise indicated.

Psilocin (PSI) synthesis was carried out following literature procedures (see Nichols, D E et al., “Improvements to the Synthesis of Psilocybin and a Facile Method for Preparing the O-Acetyl Prodrug of Psilocin” Chemistry 1999(6): 935-938, 10.1055/s-1999-3490 incorporated by reference herein in its entirety) without modifications.

2,5,8,11-tetraoxatridecan-13-yl 4-methylbenzenesulfonate (TetraEGME) synthesis was carried out following literature procedures (see 10.3390/molecules200916085, incorporated by reference herein in its entirety) without modifications.

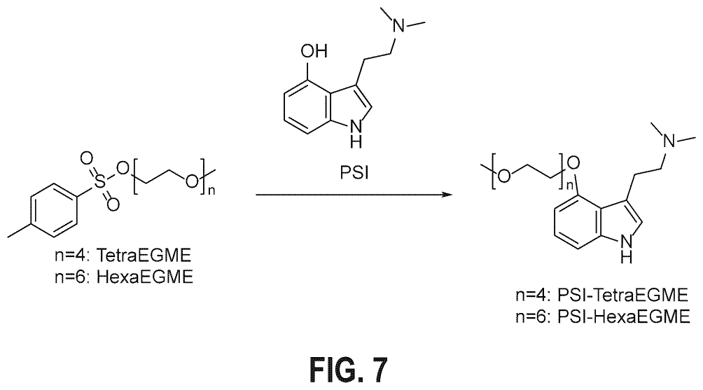

This Example 2 describes a method that was used to prepare 2-(4-((2,5,8,11-tetraoxatridecan-13-yl)oxy)-1H-indol-3-yl)-N,N-dimethylethan-1-amine (PSI-TetraEGME), as shown in FIG. 7.

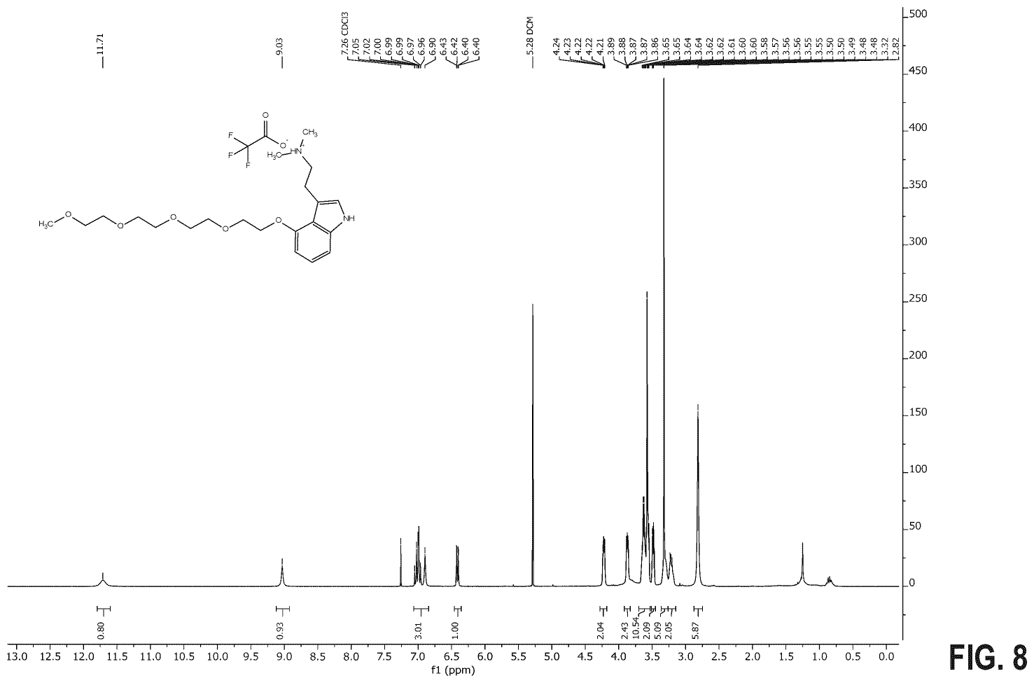

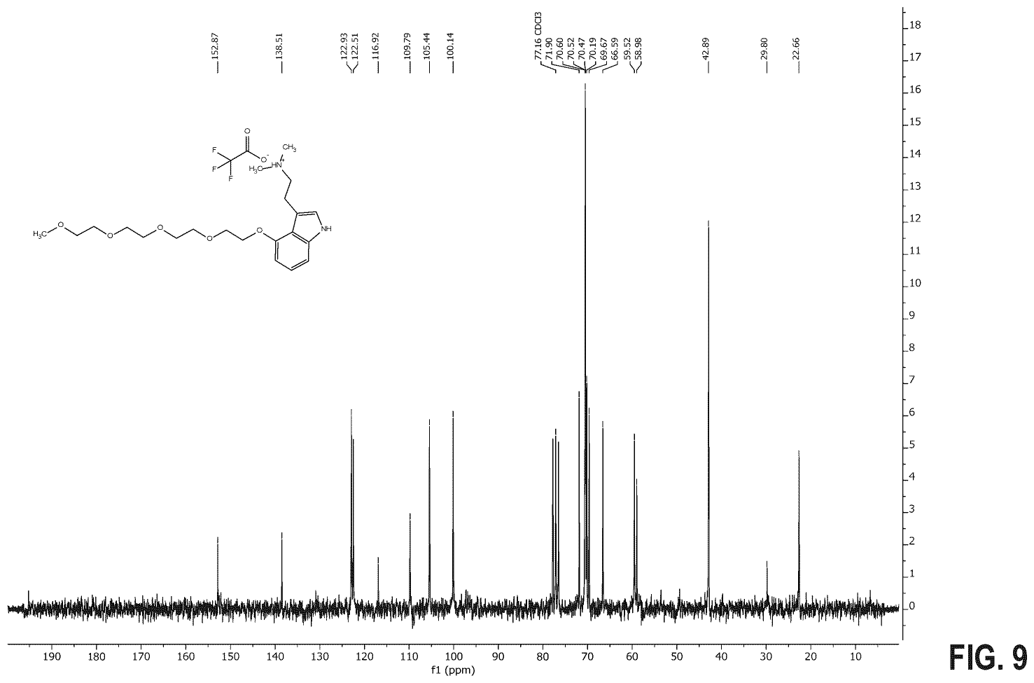

To an ice-cooled solution of 2,5,8,11-tetraoxatridecan-13-yl 4-methylbenzenesulfonate (TetraEGME, 0.150 g, 0.4 mmol) in DMF (1.35 mL) was added psilocin (PSI, 68 mg, 0.33 mmol) and Cs₂CO₃ (0.118 g, 0.36 mmol). The mixture was stirred at 50° C. for 7 h, then it was diluted with 10 ml sat. sodium bicarbonate and extracted with DCM (3×10 mL). The organic fractions were collected, dehydrated over sodium sulfate and purified via preparative RP-HPLC C-18 (eluent water+0.1% TFA/ACN, starting from 5% ACN and reaching 45% in 17 minutes, retention time 15.7 minutes), obtaining 2-(4-((2,5,8,11-tetraoxatridecan-13-yl)oxy)-1H-indol-3-yl)-N,N-dimethylethan-1-aminium trifluoroacetate (PSI-TetraEGME trifluoroacetatel (0.126 g, 0.25 mmol, 75% yield) as a brownish oil. HRMS (ESI) m/z: [M+H]⁺ Required C₂₁H₃₅N₂O₅ 395.2540; Found 395.2565. ¹H NMR (300 MHz, CDCl₃) δ 11.71 (s, 1H), 9.03 (s, 1H), 7.07-6.84 (m, 3H), 6.41 (d, J=7.2, 1.2 Hz, 1H), 4.29-4.18 (m, 2H), 3.92-3.82 (m, 2H), 3.70-3.52 (m, 10H), 3.52-3.44 (m, 2H), 3.36-3.26 (m, 5H), 3.26-3.15 (m, 2H), 2.82 (s, 6H). ¹³C NMR (50 MHz, CDCl₃) δ 152.87, 138.51, 122.93, 122.51, 116.92, 109.79, 105.44, 100.14, 71.90, 70.60, 70.52, 70.47, 70.19, 69.67, 66.59, 59.52, 58.98, 42.89, 29.80, 22.66. A graph showing the 1H NMR spectrum of PSI-TetraEGME trifluoroacetate is shown in FIG. 8, and a graph showing the 13C NMR spectrum of PSI-TetraEGME trifluoroacetate is shown in FIG. 9.

### Example 3: 2-(4-((2,5,8,11,14,17-hexaoxanonadecan-19-yl)oxy)-1H-indol-3-yl)-N,N-dimethylethan-1-amine (PSI-HexaEGME)

The compound structures in the examples below were confirmed by one or more of the following methods: ¹H NMR, ¹³C NMR, mass spectrometry, HPLC-UV. ¹H NMR spectra were determined using an NMR spectrometer operating at 300 MHz or 400 MHz field. Chemical shifts were referenced to signals from residual protons in deuterated chloroform as follows: CDCl₃=7.25 ppm. Peak multiplicities are designated as follows: s, singlet; d, doublet; t, triplet; and m, multiplet. Coupling constants are given in Hertz (Hz).

Mass spectra (MS) data are obtained using a mass spectrometer with ESI ionization and Ion trap or TOF mass analyzer.

In this and other examples herein, reagents and solvents may be purchased from commercial suppliers, and may be used without further purification unless otherwise indicated.

Psilocin (PSI) synthesis was carried out following literature procedures (see Nichols, D E et al., “Improvements to the Synthesis of Psilocybin and a Facile Method for Preparing the 0-Acetyl Prodrug of Psilocin” Chemistry 1999(6): 935-938, 10.1055/s-1999-3490 incorporated by reference herein in its entirety) without modifications.

2,5,8,11,14,17-hexaoxanonadecan-19-yl 4-methylbenzenesulfonate (HexaEGME) syntheses were carried out following literature procedures (see 10.3390/molecules200916085, incorporated by reference herein in its entirety) without modifications.

This Example 3 describes a method that was used to prepare 2-(4-((2,5,8,11,14,17-hexaoxanonadecan-19-yl)oxy)-1H-indol-3-yl)-N,N-dimethylethan-1-amine (PSI-HexaEGME), as shown in FIG. 7.

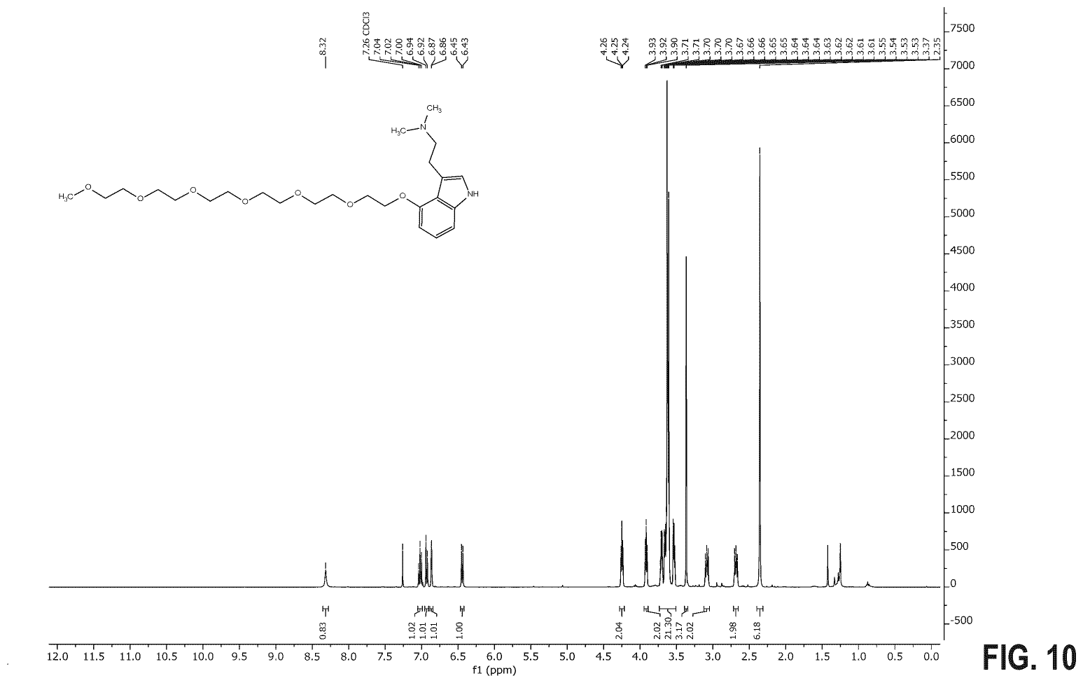

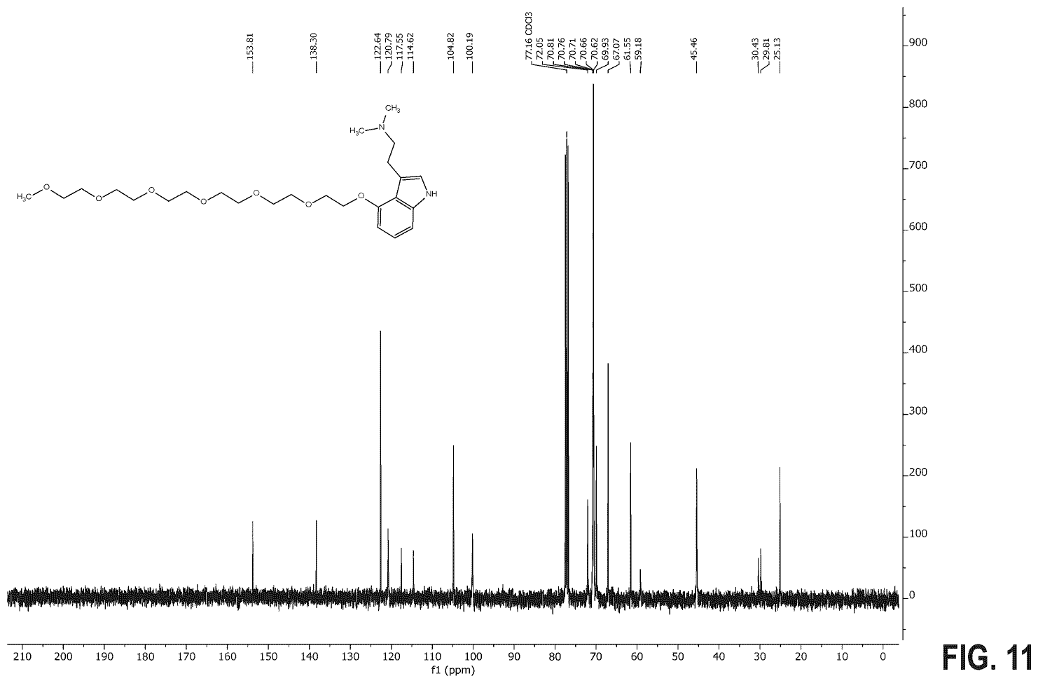

To an ice-cooled solution of 2,5,8,11,14,17-hexaoxanonadecan-19-yl 4-methylbenzenesulfonate (HexaEGME, 0.230 g, 0.52 mmol) in DMF (1.68 ml) was added psilocin (PSI, 85 mg, 0.41 mmol) and Cs₂CO₃ (0.169 g, 0.52 mmol). The mixture was stirred at 50° C. for 7 h, then it was diluted with 10 ml sat. sodium bicarbonate and extracted with DCM (3×10 mL). The organic fractions were collected, dehydrated over sodium sulfate and purified by column chromatography using CHCl₃/MeOH 8:2 as eluent system, obtaining 2-(4-((2,5,8,11,14,17-hexaoxanonadecan-19-yl)oxy)-1H-indol-3-yl)-N,N-dimethylethan-1-amine (PSI-HexaEGME), as a brownish oil (0.153 g, 0.32 mmol, 78% yield). HRMS (ESI) m/z: [M+H]⁺ Required C₂₅H₄₃N₂O₇⁺ 483.3065; Found 483.3082. ¹H NMR (400 MHz, CDCl₃) δ 8.32 (s, 1H), 7.02 (t, J=7.9 Hz, 1H), 6.93 (d, J=7.8 Hz, 1H), 6.87 (d, J=1.7 Hz, 1H), 6.44 (d, J=7.7 Hz, 1H), 4.25 (t, J=5.2 Hz, 2H), 3.92 (t, J=5.2 Hz, 2H), 3.74-3.50 (m, 20H), 3.37 (s, 3H), 3.13-3.04 (m, 2H), 2.72-2.64 (m, 2H), 2.35 (s, 6H). ¹³C NMR (101 MHz, CDCl₃) δ 153.81, 138.30, 122.64, 120.79, 117.55, 114.62, 104.82, 100.19, 72.05, 70.81, 70.76, 70.71, 70.66, 70.62, 69.93, 67.07, 61.55, 59.18, 45.46, 30.43, 29.81, 25.13. A graph showing the 1H NMR spectrum of PSI-HexaEGME is shown in FIG. 10, and a graph showing the 13C NMR spectrum of PSI-HexaEGME is shown in FIG. 11.

While the present invention has been disclosed by reference to the details of preferred embodiments of the invention, it is to be understood that the disclosure is intended as an illustrative rather than in a limiting sense, as it is contemplated that modifications will readily occur to those skilled in the art, within the spirit of the invention and the scope of the amended claims.

What is claimed is:

1. A psychedelic drug (PD) polymer conjugate of general structure PD-(X-Poly-T)ₙ wherein:
PD is a CNS active psychedelic drug targeting serotonergic receptors, and has at least one chemically reactive functional group;
n is an integer comprised between 1 and 6; and
(X-Poly-T) is, independently for each occurrence, hydrogen or the moieties are, independently for each occurrence, as follows:
X is a stable (enzymatically and/or hydrolytically under physiological conditions) linker comprising a covalent bond or a chain of atoms that covalently attaches a small molecule 5-HT receptors agonist drug moiety to the Poly derivative;
Poly is a covalently bonded chain of repeating monomer units that form a polymer or an oligomer backbone of synthetic or natural origin; and
T is terminal group of Poly and is represented by a chemical group which, depending upon preference, is unreactive or reactive with other chemical moieties, or has a targeting property.

2. The conjugate of claim 1, wherein PD is psilocin or psilocybin.

3. The conjugate in claim 1 or claim 2, wherein Poly is polyethylene glycol.

4. The conjugate of any of claims 1-3, wherein T is the methoxy group.

5. The conjugate of any of claims 1-4, wherein X is an ether group.

6. The conjugate of any of claims 1-5, wherein Poly has a molecular weight >500 Da and lower than 2000 Da.

7. The conjugate of any of claims 1-6 having a modulated ability to cross the blood brain barrier.

8. The conjugate of any of claims 1-7 for the use in treating diseases affecting the peripheral cells, said peripheral cells being cells that reside outside of the blood brain barrier.

9. The conjugate of any of claims 1-8 for use in treating diseases caused by dysfunction of immunological cells, hepatic cells, pancreatic cells, or retinal cells.

10. The conjugate of any of claims 1-9 for the treatment or prevention of autoimmune disorders.

11. A conjugate of any of claims 1-10 for the treatment or prevention of metabolic disorders, including NAFLD/NASH, diabetes, and retinopathies.

12. The conjugate of any of claims 1-11 for the treatment or prevention of a condition selected from the metabolic syndrome, obesity, hyperglycemia, type 2 diabetes mellitus, high blood pressure, coronary artery disease including myocardial infarction and unstable angina, NAFLD and NASH, hypogonadism, testosterone insufficiency, hypothalamic-pituitary axis disorders, and BDNF insufficiency, including WAGR syndrome, 11p deletion, and 11p inversion, and Prader-Willi, Smith-Magenis, and ROHHAD syndromes.

13. A pharmaceutical or diagnostic composition comprising a conjugate as defined in any of claims 1-12, optionally also comprising one or more pharmaceutically acceptable excipient.

14. The composition of claim 13 for oral, sublingual, transmucosal, intranasal, transdermal, parenteral, rectal, topical, vaginal, ophthalmic, intravitreal, corneal, or inhalation use.

15. The composition in claim 14 administered at doses ranging from 0.001 mg to 1 gram.

16. The composition of claim 1, wherein the Poly backbone is chosen from poly(ethylene glycol) (PEG), poly(N-vinylpyrrolidone), N-hydroxy-ethyl methacrylamide copolymer, poly(2-ethyl-2-oxazoline), poly(N-acryloylmorpholine), poly(propylene glycol), poly(vinyl alcohol), polyglutamic acid, hyaluronic acid, and polysialic acid and other polysaccharides.

17. The composition of claim 1, wherein Poly has an average molecular weight between 80 and 40000 Da.

18. The composition of claim 17, wherein Poly is a derivative of poly(ethylene glycol) (PEG), of linear or branched structure, mono-, bi-functional or heterobifunctional, with an average molecular weight between 120 and 40000 Da.

19. The composition of claim 1, wherein the terminal group is chosen from hydroxyl, amino, sulfide, carboxy, cyano, optionally substituted aryloxy, lower alkoxy (e.g., methoxy, ethoxy, propoxy, or butoxy), aryl, lower alkyl, lower alkenyl, lower alkynyl, cycloalkyl, halogen atom (e.g., fluorine, chlorine, bromine, iodine), tosylate, mesylate, isocyanate, hydrazine, azide, maleimide, orthopyridyl disulfide, N-succinimidyloxy, sulfo-N-succinimidyloxy, 1-benzotriazol, 1-imidazolyloxy, p-nitrophenyloxy, and formyl.
